(19) United States
(12) Patent Application Publication
(10) Pub. No.: US 2022/0387588 A1
(43) Pub. Date: Dec. 8, 2022

(54) LIPID COATED IRON OXIDE NANOPARTICLES FOR OTITIS MEDIA

(71) Applicant: Otomagnetics, Inc., Gaithersburg, MD (US)

(72) Inventors: Benjamin SHAPIRO, Redondo Beach, CA (US); Mohammed SHUKOOR, North Bethesda, MD (US); Sanjay SRINIVASAN, North Bethesda, MD (US)

(73) Assignee: Otomagnetics, Inc., Gaithersburg, MD (US)

(21) Appl. No.: 17/755,024

(22) PCT Filed: Oct. 22, 2020
(86) PCT No.: PCT/US2020/056926

§ 371 Date: Apr. 19, 2022

Related U.S. Application Data
(60) Provisional application No. 62/924,585, filed on Oct. 22, 2019.

(51) Int. Cl. A61K 41/00; A61K 9/48; A61K 9/50; A61K 31/496; A61K 31/573; A61K 9/00; A61P 27/16

(52) U.S. Cl. CPC A61K 41/00; A61K 9/485; A61K 9/4858; A61K 9/5036; A61K 31/496; A61K 31/573; A61K 9/0046; A61P 27/16

(57) ABSTRACT

A composition having nanoparticles having lipids; a polysaccharide coating, an active agent and iron oxide. The active agent can be ciprofloxacin or fluocinolone. A method of treatment of ear disease or ear infection including the administration of a pharmaceutical formulation comprising nanoparticles and magnetically pushing or pulling the nanoparticles to a treatment site.

### CROSS REFERENCE TO RELATED APPLICATIONS

This application claims the benefit of U.S. Provisional Patent Application No. 62/924,585, filed Oct. 22, 2019, which is incorporated by reference herein in its entirety.

### TECHNICAL FIELD

This application relates to compositions and nanoparticles. More specifically, this application relates to drug-alluding lipid-coated iron oxide nanoparticles.

### BACKGROUND

Otitis media-commonly referred to as chronic middle ear infection has high and frequent occurrence in pediatric populations. Otitis media (OM) is an inflammation of the middle ear. Bacterial infection accounts for a large percentage of OM cases, with more than 40% of cases attributed to Streptococcus pneumoniae infection. The current standard of care for auris formulations requires multiple administrations of drops or injections (e.g. intratympanic injections) over several days (e.g., up to two weeks), including schedules of receiving multiple injections per day.

This leads to frequent exposure of kids to antibiotics affecting their quality of life and incurring high amount of financial burden on their families. Current standard of care requires intake of oral antibiotics for up to 7 to 10 days. A strict adherence to the 10 days regimen gets quite challenging with kids and a lack of which results in inefficient treatment and relapse of infections. For frequent infections or infections that persist even after antibiotic therapy the routine practice is Tympanostomy—where a tube is inserted in the ear drum through which antibiotics are directly poured into the middle ear. Moreover, tympanostomy involves an otologic surgical procedure wherein a child is sedated under general anesthesia and often have a “bad experience” due to motor imbalance and upset stomach.

Accordingly, there is always a need for improved nanoparticles and methods for treating ear issues and ear disease. It is to these needs, among others, that this application is directed.

### SUMMARY

On aspect includes a composition comprising nanoparticles having (a) lipids; (b) a polysaccharide coating, (c) iron oxide, and (d) and an active agent. The active agent can be held in the nanoparticles with electrostatic and hydrophobic interactions. The nanoparticles can have in a length across the largest distance of less than 1000 nm. The polysaccharide coating can be carboxymethylated chitosan. The carboxymethylated chitosan can be less than 50% degree of carboxymethylation substitution. The lipids can be between 5% and 50% by weight, the iron oxide is between 20% and 60% by weight, and the polysaccharide can be between 30% and 70% by weight. The disease or condition can otitis media.

Another aspect includes a composition in which the polysaccharide is a chitosan or a chitosan-derivative polymer, wherein the chitosan-derivative polymer is selected from chitosan-PEG, N-trimethyl chitosan, or a derivative of chitosan comprising a stearic acid, cholanic acid, phthaloyl, or butyl acrylate side chain. The disease or condition is otitis media.

Another aspect includes a composition in which one or more of the nanoparticles have a maximum dimension corresponding to an undeformed droplet diameter that is less than about 1000 nm and greater than about 10 nm. The composition can have a diameter ranging from 10 to 1000 nm, and the therapeutic agent can be present in the matrix material at a concentration ranging from 0.001% to 30% by weight. The active agent can be ciprofloxacin or fluocinolone acetonide. The lipids can be cationic lipids, anionic lipid and/or phospholipids. The nanoparticles are magnetically responsive iron oxide nanoparticles.

Another aspect includes a method of treatment of ear disease or ear infection, the method comprising administering a pharmaceutical formulation comprising nanoparticles comprising iron oxide, an active agent, a polysaccharide coating, and lipids, wherein the nanoparticles carry a active agent and wherein the nanoparticles are stable from pH 2-12; and magnetically pushing or pulling the particles to a treatment site. The nanoparticles carry the therapeutic agent suitable for treatment of the ear disease or ear invention loaded within them to a patient in need thereof. The nanoparticles can have a mean particle diameter of from 1 to 600 nm. The nanoparticles can have lipids, a polysaccharide coating, and iron oxide. The composition can be administered by placing the composition proximal to an ear of the patient. The composition can be administered by placing the agent within the middle ear of the patient.

The polysaccharide coating can be a chitosan or a chitosan-derivative polymer with carboxymethylation. The polysaccharide can be a chitosan or a chitosan-derivative polymer, wherein the chitosan-derivative polymer is selected from chitosan-PEG, N-trimethyl chitosan, or a derivative of chitosan comprising a stearic acid, cholanic acid, phthaloyl, or butyl acrylate side chain.

### BRIEF DESCRIPTION OF THE DRAWINGS

The application will be more fully understood from the following detailed description taken in conjunction with the accompanying drawings in which:

FIG. 1A shows a schematic of an exemplary biopolymer (CMC, TMC) coated magnetic nanoparticle;

FIG. 1B shows a schematic of an exemplary biopolymer coated magnetic nanoparticle with anionic, cationic, and/or phospholipids;

FIG. 2 shows FTIR spectra of chitosan (blue) and carboxymethyl chitosan (orange).

FIG. 3 shows an electron microscope image of an exemplary CMC-MNP batch and a dynamic light scattering data for CMC-MNP of different sizes;

FIG. 4 shows ferrofluid-like behavior of an exemplary CMC-MNP in presence of a magnet;

FIG. 5 shows percent iron [% Fe] measured by ICP-MS for CMC-MNP transport efficiency across Caco-2 cell in the presence and absence of a magnetic field.

FIG. 6A shows percent ciprofloxacin [% Cip] delivery efficiency measured by HPLC-MS for CMC-MNP transport across Caco-2 cell in the presence and absence of a magnetic field.

FIG. 6B shows images of untreated (control) and magnetically treated (CMC-MNP) bulla cavities of guinea pigs.

FIG. 7 shows Prussian staining of tympanic membrane for magnetically treated (CMC-MNP) ears.

### DEFINITIONS

“Active ingredient” or “therapeutic agent” means the substance of a pharmaceutical drug that has therapeutic effect against the disorder to be treated, either directly or when converted in the body to the active form.

A “diluent” refers to chemical compounds that are used to dilute the therapeutic agent prior to delivery and which are biocompatible.

“Ear infection” means fungal and/or bacterial infection in the ear. The location of the infection is primarily the auditory canal. In a preferred embodiment, the term ear infection includes otomycosis, and chronic and acute otitis externa.

“A “nanoparticle” is a composition having a mean particle size (e.g., diameter) of less than 1000 nm and preferably less than 600 nm. The mean diameter can range from 1 to 500 nm or more 10-250 nm. In preferred embodiments, the mean diameter is less than 1000 nm, preferably less than 200 nm.

“Treatment” as used herein indicates any activity that is part of a medical care for or deals with a condition medically or surgically. “Treat,” “treating” or “treatment” include alleviating, abating or ameliorating a disease or condition, for example tinnitus, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

“Polymer” indicates a large molecule composed of repeating structural units typically connected by covalent chemical bonds. A suitable polymer may be a linear and/or branched, and can take the form of a homopolymer or a co-polymer. If a co-polymer is used, the co-polymer may be a random copolymer or a branched co-polymer. Exemplary polymers comprise water-dispersible and in particular water-soluble polymers. For example, suitable polymers include, but are not limited to polysaccharides, polyesters, polyamides, polyethers, polycarbonates, polyacrylates, etc. For therapeutic and/or pharmaceutical uses and applications, the polymer should have a low toxicity profile and in particular that are not toxic or cytotoxic. Suitable polymers include polymers having a molecular weight of about 500,000 or below. In particular, suitable polymers can have a molecular weight of about 100,000 and below.

### DETAILED DESCRIPTION

This application discloses a non-invasive method capable of delivering drugs efficiently across the ear drum or through tissue. The method includes a more direct and local treatment, which includes the use a composition including nanoparticles having lipids, a polysaccharide coating, and iron oxide. This method can be non-invasive and requires minimal or no surgical intervention or any manipulation of perforations of the eardrum. The compositions provide a drug delivery platform which uses an external magnetic field to deliver a biodegradable, biocompatible, non-toxic and safe-magnetic nanoparticles to deliver therapeutics (e.g., antibiotics/steroids) across the tympanic membrane into the middle ear cavity, non-invasively and in limited time period.

Specific embodiments include a composition or a pharmaceutical formulation comprising nanoparticles. The nanoparticles carry a therapeutic agent or multiple therapeutic agents suitable for treatment of a disorder loaded within them and for use in the treatment of the disorder. The disorder can be ear infections and ear disease.

The nanoparticles include are diamagnetic, superparamagnetic, ferromagnetic or ferrimagnetic nanoparticles. In some embodiments, the nanoparticles are iron oxide ferrimagnetic nanoparticles.

In one embodiment, the composition includes magnetic nanoparticles carrying drugs (e.g., ciprofloxacin and steroids). One example includes nanoparticles having ciprofloxacin and steroids. Under the influence of a magnetic field, drug carrying magnetic nanoparticles can efficiently cross the tympanic membrane and delivered the drugs effectively into the desired (target site)-middle ear cavity.

In one embodiment, the formulation or compositions for treating ear infections comprises active ingredients for treating fungal and bacterial infections of ciprofloxacin and steroids, a magnetic material, and an otically acceptable carrier that can be in fluid form and release the active ingredient or agent. The formulation may be administered as a single dose to a subject's or patient's ear. The magnetic material can be selected from the group consisting of magnetic iron oxides.

In another embodiment, a nanoparticle comprises magnetic iron oxide cores in a wide size range (2-50 nm) embedded in a biopolymer (e.g., carboxymethyl chitosan (CMC) or trimethylene chitosan (TMC) biopolymers). The CMC magnetic nanoparticles sizes can be tuned based on intended applications and exemplary particles range in size from 50-450 nm in diameter. Further, the surface charge of the exemplary nanoparticles can be selected and is dependent on the type if polymer coating. The exemplary nanoparticles can carry a wide range of therapeutics (antibiotics, genes, oligonucleotides, steroids, etc.) without a need of any chemical or covalent bonding or modification of payload(s).

The magnetic nanoparticles can be coated by a coating that stabilizes the magnetic nanoparticles in the composition, e.g., to facilitate its administration. The coating may also prevent the aggregation of the magnetic nanoparticles.

In one embodiment, the amount of lipids, iron oxide, and polysaccharide can be selected to optimize the transmission of the nanoparticles through tissue or ear tissue. In one example, the amount of polysaccharide is between 20% and 90% by weight, or between 30% and 70% by weight or between 30% and 50% by weight. In another example, the amount of lipids is between 2% and 70% by weight, or between 5% and 50% by weight or between 10% and 30% by weight. In another example, the amount of iron oxide is between 5% and 80% by weight, or between 20% and 60% by weight or between 10% and 50% by weight.

In another embodiment, a nanoparticle comprises magnetic iron oxide cores in a wide size range (2-50 nm) embedded in CMC or TMC. CMC-MNP sizes can be tuned based on intended applications and exemplary particles range in size from 50-450 nm in diameter. Further, the surface charge of the exemplary nanoparticles can be selected and is dependent on the type if polymer coating. The active ingredient therein can be ciprofloxacin and/or steroids.

In another embodiment CMC is carboxymethylated or undergoes carboxymethylation, e.g., between 5 and 60% degree of substitution. In another embodiment, the degree of substitution of CMC is between 30% and 50%.

The actual dosage levels of the active ingredient or agent in the pharmaceutical compositions herein described may be varied so as to obtain an amount of the active ingredient which is effective to achieve the desired therapeutic response for a particular individual, composition, and mode of administration, without being toxic to the individual or patient.

In one embodiment, the particles may be push or pulled from the site to a treatment site by, e.g., magnetic forces including, e.g., devices disclosed in U.S. Patent Publication No. 20100212676. In one example, the composition can be administered into or near the tissue to be treated, i.e. preferentially less than 50, 40, 30, 20, 10, 5 or 1 cm away from the tissue volume and then push or pulled to the site of treatment.

In one embodiment, the formulations are composed of nanoparticles. The first nanoparticle cores or sizes each independently can have a size ranging from about 1 nm to 600 nm. In other embodiments, the nanoparticle in any of the various forms each independently have a size of at least 1 nm, 2 nm, 5 nm, 10 nm, 20 nm, 50 nm, or 100 nm and/or up to 10 nm, 20 nm, 50 nm, 100 nm, 200 nm, 500 nm, or 600 nm (i.e., the first and second nanoparticle sizes can be the same or different). Nanoparticle size can represent nanoparticle diameter or other characteristic dimension, for example an average size such as weight-, number-, or volume-average of a distribution of nanoparticles, whether for metal nanoparticles, metal-containing nanoparticles, polymer nanoparticles, polymer-containing nanoparticles, magnetic nanoparticles, or otherwise. Magnetic iron oxide particles in desirable size ranges and with excellent colloidal stability are made in the presence of customizable CMC.

In further embodiments, the compositions can be applied alone or as a carrier for active ingredients (e.g., antibiotics, anti-fungal, cortical steroids, non-steroidal anti-inflammatories, anti-parasites, and the like).

### Preparation

Lipid modified CMC and TMC derivatives are prepared by mixing an aqueous solution of purified and dried CMC or TMC with an ethanolic solution of lipids (cationic or neutral or anionic). Lipids and phospholipids (cationic, anionic, zwitterionic) alter the permeability of stratum corneum (outermost layer of tympanic membrane) by modifying its solubility or partition coefficient. Lipid coating of nanocarriers improves their cellular delivery by interacting with the lipid regions of intercellular bilayers in stratum corneum.

The mixture can then left to mix at room temperature or cold conditions or high temperature for 1 H to 24 H. Finally, CMC-lipid (or TMC-lipid) mixture can be precipitated by adding alcohol and washed off excess reagents. The product is dried under ambient conditions to obtain a free-flowing powder. Chitosan-a naturally existing carbohydrate polymer with excellent biocompatibility, biodegradability, and low toxicity is commercially available but its biological/medical applications are limited as its soluble in acidic pH only (pH<3.5).

Lipids includes cationic (DOTAP, DOTMA, DORIE, and others), anionic (DOPG), phospholipids (phosphatidylcholines, phosphatidylserine, and others). CMC polymer is further modified with lipids and phospholipids for preparing magnetic particles with enhanced tissue penetration properties.

In one example, a nanoparticle with CMC having a solubility range from pH 2.0 to 13.0 by controlling the degree of carboxymethylation and size of the parent material (chitosan). In order to improve its solubility derivatives of chitosan (O-carboxymethyl chitosan (O-CMC), N,O-carboxymethyl chitosan (N,O-CMC), N-Trimethyl chitosan (TMC), are prepared. Commercially available CMC has either high molecular weight or have a narrow solubility range. These derivatives are unsuitable for synthesis of iron oxide particles which requires a pH range from 2.5 to 12.0.

The synthesis of biopolymer-lipid coated magnetic nanoparticles (CMC-lipid-MNP and TMC-lipid-MNP) can be prepared using an illustrative method. In this methodology, the CMC can be carboxymethylated. Excessive and inadequate amounts of carboxymethalation lead to a collapsing nanoparticle. An exemplary CMC-lipid magnetic nanoparticle (CMC-lipid MNP) can be developed via two methods; (i) Co-precipitation of iron oxide cores in the presence of biopolymer and (ii) Post functionalization of preformed iron oxide cores.
(i) One-step co-precipitation: In a typical procedure a stoichiometric mixture of Fe (II) and Fe (III) salts was precipitated in the presence of CMC-lipid by addition of an alkaline solution under inert conditions. The solution was heated to 80C and allowed to stir for 1 h before cooling down to room temperature. The resulting CMC-lipid-MNP solution was washed with water by stirred filtration to remove excess of reagents.
(ii) Two-step post functionalization: In this method pre-synthesized iron oxide nanoparticles were coated with the above prepared biopolymer derivatives. In a typical procedure, 10 mg of iron oxide cores were dissolved in 10 ml aqueous solution containing 50 mg of CMC-lipid. The mixture was sonicated for 20 min and was left to stir for 12 h at room temperature to ensure proper coating of iron oxide cores. The resulted black colloidal solution was purified by stirred filtration technique several times in order to remove free CMC-lipid.

In one embodiment, the active agent can be loaded into the nanoparticles. In one embodiment, between 0.001% and 30% by weight of the active agent can be loaded into the nanoparticles. In one embodiment, between 0.1% and 30% by weight of the active agent can be loaded into the nanoparticles. In one embodiment, between 0.1% and 20% by weight of the active agent can be loaded into the nanoparticles. For example, between 0.1% to 30% of an antibiotic can be loaded into the nanoparticles. In other examples, between 0.1% and 20% of steroid can be loaded into the nanoparticles.

In some embodiments, example active agent or ingredients for use within the nanoparticles are antibiotics including aminoglycosides, amikacin, gentamicin, kanamycin, neomycin, netilmicin, tobramycin, paromomycin, spectinomycin, ansamycins, geldanamycin, herbimycin, rifaximin, carbacephem, loracarbef, carbapenems, ertapenem, doripenem, meropenem, cephalosporins, cefadroxil, cefazolin, cefalotin, cefalexin, cephalosporins, cefaclor, cefamandole, cefoxitin, cefprozil, cefuroxime, cephalosporins, cefixime, cefdinir, cefditoren, cefoperazone, cefotaxime, cefpodoxime, ceftazidime, ceftibuten, ceftizoxime, ceftriaxone, cephalosporins, cefepime, cephalosporins, ceftaroline fosamil, ceftobiprole, glycopeptides, teicoplanin, vancomycin, telavancin, lincosamides, clindamycin, lincomycin, lipopeptide, daptomycin, macrolides, azithromycin, clarithromycin, dirithromycin, erythromycin, roxithromycin, troleandomycin, telithromycin, spiramycin, monobactams, aztreonam, nitrofurans, furazolidone, nitrofurantoin, oxazolidonones, linezolid, posizolid, radezolid, torezolid, penicillins, amoxicillin, ampicillin, azlocillin, carbenicillin, cloxacillin, dicloxacillin, flucloxacillin, mezlocillin, methicillin, nafcillin, oxacillin, penicillin, piperacillin, temocillin, ticarcillin, polypeptides, bacitracin, colistin, polymyxin b, quinolones, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, sulfonamides, mafenide, sulfadiazine, sulfamethizole, sulfamethoxazole, sulfanilimide (archaic), sulfasalazine, sulfisoxazole, tetracyclines, demeclocycline, doxycycline, minocycline, oxytetracycline, tetracycline, clofazimine, dapsone, capreomycin, cycloserine, ethambutol, ethionamide, isoniazid, pyrazinamide, rifabutin, rifapentine, streptomycin, arsphenamine, chloramphenicol, fosfomycin, metronidazole, mupirocin, quinupristin/dalfopristin, thiamphenicol, tigecycline, trimethoprim, and combinations thereof, among others.

In other embodiments, example active ingredients for use within the pharmaceutical compositions as suitable antifungals include amrolfine, utenafine, naftifine, terbinafine, flucytosine, fluconazole, itraconazole, ketoconazole, posaconazole, ravuconazole, voriconazole, clotrimazole, econazole, miconazole, oxiconazole, sulconazole, terconazole, tioconazole, nikkomycin Z, caspofungin, micafungin, anidulafungin, amphotericin B, liposomal nystastin, pimaricin, griseofulvin, ciclopirox olamine, haloprogin, tolnaftate, undecylenate, clioquinol, and combinations thereof, among others.

In some embodiments, example active ingredients for use within the pharmaceutical compositions as cortical steroids include hydrocortisone, prednisone, fluprednisolone, triamcinolone, dexamethasone, betamethasone, cortisone, prednilosone, methylprednisolone, fluocinolone acetonide, flurandrenolone acetonide, and fluorometholone, among others.

The dosage of the therapeutic agent may be selected depending on the desired administration dose to the patient, and may vary depending on the agent to be used, and the composition of the nano and microparticles.

Provided herein are pharmaceutical compositions that include at least one active agent and a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In other embodiments, the pharmaceutical compositions also contain other therapeutic substances.

In some embodiments, the compositions are formulated for pH, and a practical osmolality or osmolarity to help promote that homeostasis of the target auris structure is maintained. A perilymph-suitable osmolarity/osmolality is a practical/deliverable osmolarity/osmolality that maintains the homeostasis of the target auris structure during administration of the pharmaceutical formulations described herein.

In some embodiments, the otic disease or condition is otitis externa, otitis media, Ramsay Hunt syndrome, otosyphilis, AIED, Meniere's disease, or vestibular neuronitis.

Further details concerning the identification of the suitable carrier agent or auxiliary agent of the compositions, and generally manufacturing and packaging of the kit, can be identified by the person skilled in the art upon reading of the present disclosure.

In some embodiments, the active agent is an immediate release agent. In some embodiments, the active agent is a controlled release agent.

In some embodiments of the methods described above, the active agent is released from the composition for a period of at least 1, 2, 3, 4 or 5 days. In some embodiments, the active agent may be released from the composition for a period of at least 5 or 7 days.

In some embodiments, a composition disclosed herein is administered to an individual in need thereof once. In some embodiments, a composition disclosed herein is administered to an individual in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein. In some embodiments, a first administration of a composition disclosed herein is followed by a drug holiday.

### EXAMPLES

### Example 1: Significance of Biopolymer Coated Magnetic Nanoparticles

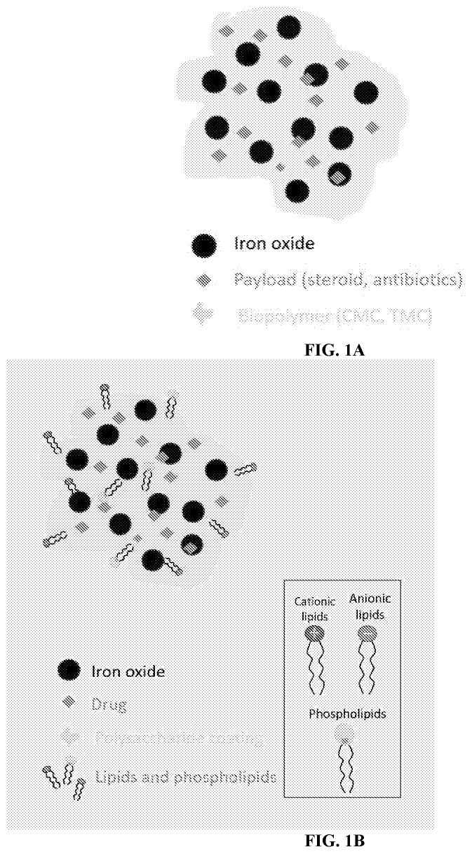

FIG. 1A illustrates one schematic version of a particles. The particles, for safely and effectively traversing tissue barriers under the action of an applied magnetic gradient, are composed of biodegradable and biocompatible polysaccharides such as chitosan derivatives (O-carboxymethyl chitosan (O-CMC), N,O-carboxymethyl chitosan (N,O-CMC), N-Trimethyl chitosan (TMC), and others). Chitosan derivatives such as CMC and TMC are non-toxic, safe, easy to prepare and scale up and are widely reported as efficient absorption enhancer in drug delivery across cells and tissue.

### Example 2: Exemplary CMC Magnetic Nanoparticles (CMC-MNP)

FIG. 1B show that the nanoparticles consist of magnetic iron oxide cores in a wide size range (2-50 nm) embedded in a customizable biopolymer (CMC or TMC). The CMC-MNP sizes can be tuned based on intended applications and exemplary particles range in size from 50-450 nm in diameter. Further, the surface charge of the exemplary nanoparticles can be easily selected and is dependent on the type if polymer coating. The exemplary nanoparticles can carry a wide range of therapeutics (antibiotics, genes, oligonucleotides, steroids, etc.) without a need of any chemical or covalent bonding or modification of payload/s.

### Example 3

Synthesis of Biopolymer derivatives (CMC and TMC). CMC synthesis: Carboxymethylation of chitosan is achieved by pretreating chitosan with an alkali solution followed by reacting with chloroacetic acid in the presence of isopropanol/water as a solvent mixture. Reaction time and reagent concentration (chloroacetic acid and sodium hydroxide) were varied to control the degree of substitution.

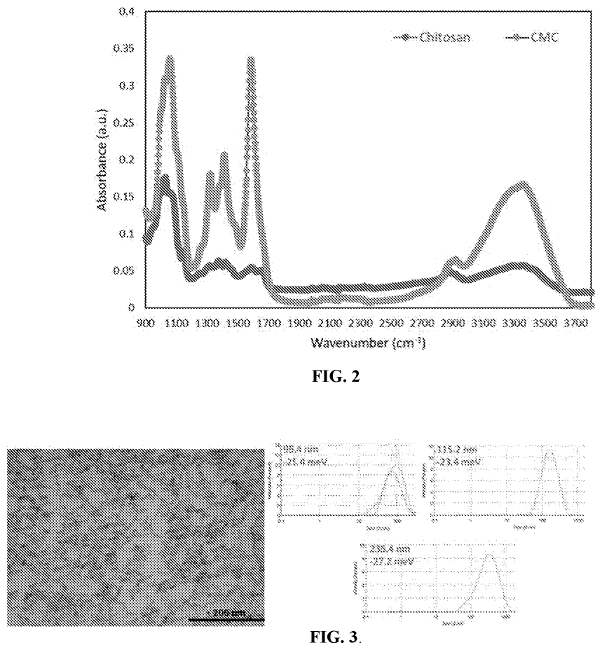

Fourier transformed-infra red (FT-IR) spectra (FIG. 2) for the starting material chitosan (blue) and in-house prepared CMC were analyzed to determine the structural changes in response to the carboxymethylation process. Both spectra showed a broad absorption band between 3450 cm-1 and 3100 cm-1, due to O—H stretching vibrations and N—H extension vibration, respectively. The band at 2879 could be assigned to ν(C—H) from CH2 groups. The asymmetric stretching vibration of the carboxylate group appears around 1600 cm-1 (overlaps with N—H bend) and carboxymethyl group at 1401 cm-1. The increase intensity of peaks at 1599 cm_1 and 1324 cm_1 for CMC is an indication of carboxymethylation on both the amino and hydroxyl groups of chitosan.

TMC synthesis: TMC is prepared directly by reacting chitosan and methyl iodide under strong basic conditions at room temperature. N-methyl-2-pyrrolidone (NMP) was used as a solvent.

### Example 4

Synthesis of biopolymer coated magnetic nanoparticles (CMC-MNP and TMC-MNP) The exemplary CMC magnetic nanoparticles (CMC-MNP) can be developed via two methods; (i) Co-precipitation of iron oxide cores in the presence of biopolymer and (ii) Post functionalization of preformed iron oxide cores.
(iii) One-step co-precipitation: In a typical procedure a stoichiometric mixture of Fe (II) and Fe (III) salts was precipitated in the presence of CMC by addition of an alkaline solution under inert conditions. The solution was heated to 80C and allowed to stir for 1 h before cooling down to room temperature. The resulting CMC-MNP solution was washed with water by stirred filtration to remove excess of reagents.
(iv) Two-step post functionalization: In this method pre-synthesized iron oxide nanoparticles were coated with the above prepared biopolymer derivatives. In a typical procedure, 10 mg of iron oxide cores were dissolved in 10 ml aqueous solution containing 50 mg of CMC. The mixture was sonicated for 20 min and was left to stir for 12 h at room temperature to ensure proper coating of iron oxide cores. The resulted black colloidal solution was purified by stirred filtration technique several times in order to remove free CMC.

### Example 5: Drug Loading of CMC-MNP

For an exemplary nanoparticles antibiotic and steroid were simultaneously loaded via electrostatic and hydrophobic interactions between the CMC-MNP and drugs. In a typical method, a mixture of Ciprofloxacin (Cip) and Fluocinolone acetonide (FA) was prepared in a cocktail of propyleneglycol:glycerol:water. The CMC-MNP was mixed with the Cip+FA+cocktail for 10-15 min at ambient conditions. No washing step was required.

### Example 6 Characterization of CMC-MNP

Both above methods resulted in magnetic nanoparticles with similar physiochemical properties. The nanoparticles size can be customized based on intended applications and exemplary particles range in size from 50-450 nm in diameter.

FIG. 3 (A) shows an electron microscope image and FIG. 3 (B) shows dynamic light scattering data for CMC-MNP prepared in different sizes by the co-precipitation method. Exemplary CMC-MNP exhibit a capability to encapsulate magnetic cores of various sizes (from 2-50 nm in size, e.g. 5 nm, 10 nm, or 20 nm in size) while keeping the final particle size <450 nm.

### Iron Loading

Fe content corresponding to the concentration of iron-oxide cores in exemplary CMC-MNP was characterized by ICP-MS. CMC-MNP contains iron loading of 35% [Fe]=0.06-0.40 mg (iron)/mg (CMC-MNP).

### Drug Loading

Ciprofloxacin loading was quantified using standard HPLC-MS (high performance liquid chromatography mass spectrometry). Cip-loaded CMC-MNP were precipitated in methanol:0.1% HCl V/V (1:1) solvent mixture followed by centrifugation. The supernatant was submitted for analysis. A drug loading of 10-30% was achieved for current nanoparticles.

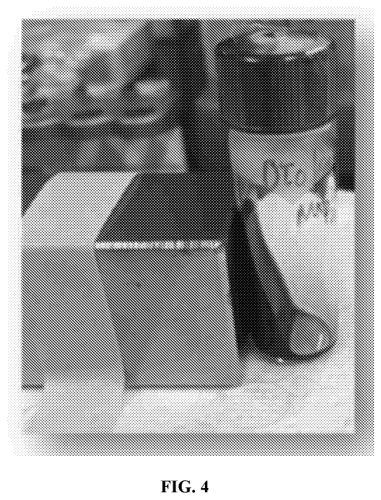

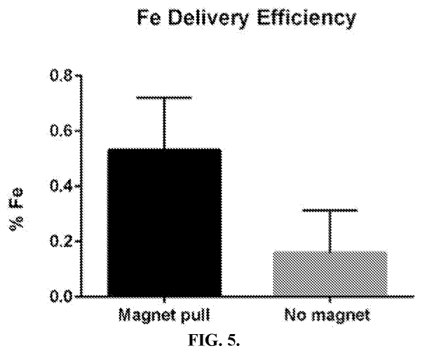

The CMC-MNP behaves like a ferrofluid in its final form. FIG. 4 shows a solution of the exemplary CMC-MNP in a glass vial being pulled towards a magnet (0.4 T).

### Example 7: Biophysical Characterization of CMC-MNP

The transport of CMC-MNP in the presence of an external magnetic field through biological barriers was tested, both, in vitro -using different cell lines and in vivo—in live animals.
(i) CMC-MNP transport efficiency across cell layers

In a first set of studies, CMC-MNP were incubated with epithelial cells (Caco-2 and T84) grown on transwell system. A magnetic gradient was applied with a pull magnet (0.4 T) to test the transport efficiency of nanoparticles through the cell layer into the bottom receiving chamber. The transport efficiency of MNP across the cell layers in response to the magnetic force was quantitated by measuring the [Fe] by ICP-MS or ICP-OES (inductively coupled plasma mass-spectrometry or optical emission spectrometry). As seen in FIG. 5 the transport efficiency of CMC-MNP was five-fold higher in the presence of an external magnetic field than in the absence magnet.
(ii) Drug delivery efficiency across cell layers

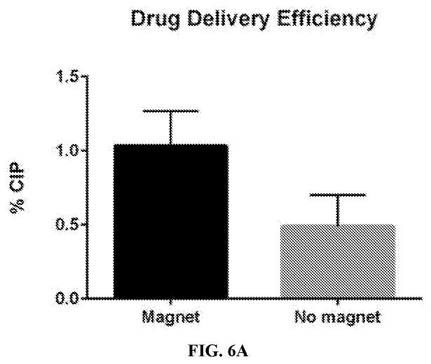

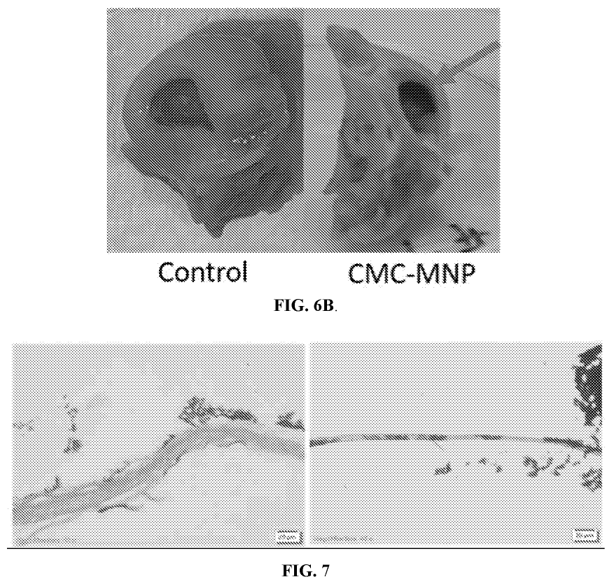

Using the similar experimental setup as described above drug delivery efficiency was measured across the cell layers using HPLC-MS. Cip-loaded CMC-MNP were prepared as described above (Example 4). FIG. 6A exhibits the drug delivery efficiency of CMC-MNP through cell layers in the presence and absence of an external magnetic field. As seen Cip delivery was doubled in the presence of magnet due to efficient delivery of CMC-MNP.

### Example 8: In Vivo Studies

For in vivo studies CMC-MNP were placed in the outer ear canal of guinea pigs, and then a magnetic gradient was applied with a pull device to test the motion of nanoparticles across the ear drum (the tissue barrier) into the middle ear tissues (the target). FIG. 6B shows the bulla cavity excised from untreated and magnetically administered CMC-MNP guinea pigs. Nanoparticle treated bulla shows intense black stain (red arrow) inside the cavity due to accumulation of iron oxide nanoparticles.

Furthermore, Prussian blue staining was performed to qualitatively assess the presence of iron (from iron oxide nanoparticles) in the target tissue. FIG. 7 shows Prussian staining of tympanic membrane for magnetically treated (CMC-MNP) ears. Iron oxide nanoparticles in great abundance can be visualized throughout the tympanic membrane and to some extent in the middle ear space. This verifies that the nanoparticles could traverse the ear drum and entered the targeted middle ear space.

### References

1. Berman S. Otitis media in children. N Engl J Med. 1995;332(23):1560-1565

2. Todberg T, Koch A, Andersson M, Olsen S F, Lous J, Homoe P. PLoS One. 2014;9(12):e111732

3. Lanphear B P, Byrd R S, Auinger P, Hall C B. Pediatrics. 1997;99(3):E1

4. Bondy J, Berman S, Glazner J, Lezotte D. Pediatrics. 2000;105(6):E72.

5. McCaig L F, Besser R E, Hughes J M. JAMA. 2002;287(23):3096-102

6. Lieberthal AS, et al. Pediatrics. 2013;131(3):e964-e999

7. (a) Wang J, Kong M, Zhou Z, Yan D, Yu X, Cheng X, Feng C, Liu Y, Chen X. Carbohydr Polym. 2017;157:596-602. (b) Benediktsdóttir B E, Gudjónsson T, Baldursson Ó, Másson M J. Eur J Pharm Biopharm. 2014;86(1):55-63

8. Thanou M, Verhoef J C, Junginger H E. Adv Drug Deliv Rev. 2001;52(2):117-26.

The examples set forth above are provided to give those of ordinary skill in the art a complete disclosure and description of how to make and use the embodiments of the particles, compositions, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

Although any methods and materials similar or equivalent to those described herein can be used in the practice for testing of the specific examples of appropriate materials and methods are described herein.

A number of embodiments of the disclosure have been described. Nevertheless, it will be understood that various modifications may be made without departing from the spirit and scope of the present disclosure. Accordingly, other embodiments are within the scope of the following claims.

1. A composition comprising nanoparticles having (a) lipids; (b) a polysaccharide coating, (c) iron oxide, and (d) and an active agent, wherein the active agent is held in the nanoparticles with electrostatic and hydrophobic interactions.

2. The composition of claim 1, wherein the nanoparticles have in a length across the largest distance of less than 1000 nm.

3. The composition of claim 1, wherein the polysaccharide coating is carboxymethylated chitosan.

4. The composition of claim 3, wherein the carboxymethylated chitosan has less than 50% degree of carboxymethylation substitution.

5. The composition of claim 1, wherein the lipids are between 5% and 50% by weight, the iron oxide is between 20% and 60% by weight, and the polysaccharide is between 30% and 70% by weight.

6. The composition of claim 1, wherein the polysaccharide is a chitosan or a chitosan-derivative polymer, wherein the chitosan-derivative polymer is selected from chitosan-PEG, N-trimethyl chitosan, or a derivative of chitosan comprising a stearic acid, cholanic acid, phthaloyl, or butyl acrylate side chain.

7. The composition of claim 1, wherein the otic disease or condition is otitis media.

8. The composition, wherein one or more of the nanoparticles have a maximum dimension corresponding to an undeformed droplet diameter that is less than about 1000 nm and greater than about 10 nm.

9. The composition of claim 10, wherein the composition has a diameter ranging from 10 to 1000 nm, and the therapeutic agent is present in the matrix material at a concentration ranging from 0.001% to 30% by weight.

10. The composition of claim 1, wherein the active agent is ciprofloxacin.

11. The composition of claim 1, wherein the active agent is fluocinolone acetonide.

12. The composition of claim 1, wherein the lipids are cationic lipids.

13. The composition of claim 1, wherein the lipids are anionic lipids.

14. The composition of claim 1, wherein the lipids are phospholipids.

15. The composition of claim 1, wherein the nanoparticles are magnetically responsive iron oxide nanoparticles.

16. The composition of claim 1, wherein the lipids are cation lipids.

17. A method of treatment of ear disease or ear infection, the method comprising administering a pharmaceutical formulation comprising nanoparticles comprising iron oxide, an active agent, a polysaccharide coating, and lipids, wherein the nanoparticles carry an active agent and wherein the nanoparticles are stable from pH 2-12; and magnetically pushing or pulling the particles to a treatment site, wherein the nanoparticles carry the therapeutic agent suitable for treatment of the ear disease or ear invention loaded within them to a patient in need thereof .

18. The method of claim 17, wherein the nanoparticles have a mean particle diameter of from 1 to 600 nm.

19. The method of claim 17, wherein the nanoparticles comprise lipids, a polysaccharide coating, and iron oxide.

20. The method of claim 17, wherein the polysaccharide coating is a chitosan or a chitosan-derivative polymer with carboxymethylation.

21. The method of claim 17, wherein the polysaccharide is a chitosan or a chitosan-derivative polymer, wherein the chitosan-derivative polymer is selected from chitosan-PEG, N-trimethyl chitosan, or a derivative of chitosan comprising a stearic acid, cholanic acid, phthaloyl, or butyl acrylate side chain.

22. The method of claim 17, wherein the composition is administered by placing the composition proximal to an ear of the patient.

23. The method of claim 17, wherein the composition is administered by placing the agent within the middle ear of the patient.

24. The method of claim 17, wherein the active agent is ciprofloxacin.

25. The method of claim 1, wherein the active agent is fluocinolone acetonide.
